(19) United States
(12) Patent Application Publication
(10) Pub. No.: US 2008/0107722 A1
(43) Pub. Date: May 8, 2008

(54) Method For Loading Multiple Agents Into Delivery Vehicles

(76) Inventors: Paul Tardi, Surrey (CA); Murray Webb, North Vancouver (CA); Sharon Johnstone, Vancouver (CA); Pierrot Harvie, Vancouver (CA); Lawrence Mayer, North Vancouver (CA)

Correspondence Address: MORRISON & FOERSTER LLP, 12531 HIGH BLUFF DRIVE, SUITE 100, SAN DIEGO, CA, 92130-2040, US

(21) Appl. No.: 11/719,088

(22) PCT Filed: Nov. 18, 2005
(86) PCT No.: PCT/US05/42131

§ 371 Date: Oct. 9, 2007

Related U.S. Application Data
(60) Provisional application No. 60/629,501, filed on Nov. 18, 2004.

(51) Int. Cl. A61K 9/127

(52) U.S. Cl. 424/450

(57) ABSTRACT

This invention relates to encapsulation of drugs and other agents into preformed delivery vehicles.

### RELATED APPLICATION

This application claims benefit of U.S. application Ser. No. 60/629,501 filed 18 Nov. 2004 which is incorporated herein by reference in its entirety.

### TECHNICAL FIELD

The invention relates to a method for loading a lipid carrier with multiple agents and to the lipid-based carrier compositions formed thereby. More particularly, the invention concerns a method which ensures the simultaneous encapsulation of therapeutic agents wherein at least one therapeutic agent is passively loaded and at least a second therapeutic agent is actively loaded into preformed liposomes.

### BACKGROUND ART

The progression of many life-threatening diseases such as cancer, AIDS, infectious diseases, immune disorders and cardiovascular disorders is influenced by multiple molecular mechanisms. Due to this complexity, achieving cures with a single agent has been met with limited success. Thus, combinations of agents have often been used to combat disease, particularly in the treatment of cancers. It appears that there is a strong correlation between the number of agents administered and cure rates for cancers such as acute lymphocytic leukemia (Frei, et al., Clin. Cancer Res. (1998) 4:2027-2037). Clinical trials utilizing combinations of doxorubicin, cyclophosphamide, vincristine, methotrexate with leucovorin rescue and cytarabine (ACOMLA) or cyclophosphamide, doxorubicin, vincristine, prednisone and bleomycin (CHOP-b) have been successfully used to treat histiocytic lymphoma (Todd, et al., J. Clin. Oncol. (1984) 2:986-993). Furthermore, the effects of combinations of drugs are enhanced when the ratio in which they are supplied provides a synergistic effect. Synergistic combinations of agents have also been shown to reduce toxicity due to lower dose requirements, to increase cancer cure rates (Barriere, et al. Pharmacotherapy (1992) 12:397-402; Schimpff, Support Care Cancer (1993) 1:5-18), and to reduce the spread of multi-resistant strains of microorganisms (Shlaes, et al. Clin. Infect. Dis. (1993) 17:S527-S536).

Conventional combination therapies involve the administration of free drug “cocktails”. Drugs are typically utilized at the maximum tolerated dose (MTD) of each drug and administered at the same time or in sequence. A major disadvantage to this technique is that there is no mechanism for controlling the dissimilar pharmacokinetics of the individual drugs and therefore the concentration of components in the cocktail which reaches the target tissue may not be the same as that which is administered. This is important because it is now known that whether drug combinations act synergistically or antagonistically to kill target cells is dependent upon the ratio and concentration of the drugs present (see WO 03/028696). Consequently, administration of drug combinations as uncontrollable aqueous mixtures or cocktails no doubt leads to sub optimal drug ratios with a resulting loss of therapeutic activity. Controlling the ratio of agents after systemic administration is particularly important if the agents have been combined to achieve a synergistic effect given this dependency of synergy on drug:drug ratios. One would aim therefore to better control the systemic concentrations, and thus ratio, of the combined components in order to achieve optimal therapeutic activity. In recent years, researches have taken advantage of pharmaceutical carriers, such as liposomes, to better control the delivery of drugs.

Lipid-based delivery vehicles are known to improve the therapeutic index of a variety of drugs by ameliorating toxicity and/or increasing therapeutic potency of the encapsulated agent. This is perhaps best exemplified in the delivery of anticancer drugs where it has been well documented both preclinically and clinically that small (approx. 100 nm) liposomes reduce exposure of entrapped drugs to susceptible healthy tissues while preferentially accumulating at sites of tumor growth due to enhanced permeability and retention (EPR) effects. This in turn has often resulted in improvements in the overall therapeutic activity of the drug and has led to the regulatory approval of several liposome-based anticancer products. Interestingly, very little work has been undertaken to deliver drug combinations in liposomes. This is likely the result of difficulties associated with the efficient and stable encapsulation of two therapeutic agents inside a single liposome as well as challenges of controlling the release of chemically disparate drugs with one liposomal composition. Furthermore, the pharmacokinetics of currently available formulations vary greatly which would hinder attempts to coordinate the exposure of different drugs using a combination of existing liposome formulations. Combining existing liposome formulations poses even greater difficulties since this potentially would lead to high lipid doses which have been shown to cause infusion-related adverse events in humans. Therefore, a need exists to not only control the delivery of a combination of therapeutic agents but also to minimize the amount of lipid required to do so.

In utilizing liposomes for delivery it is generally desirable to load the therapeutic agents to a high encapsulated-concentration. Classical means of entrapping or loading agents within liposomes included passively encapsulating the desired agent during the preparation of the liposomes (for examples see: Deamer, et al., Biochim. Biophys. Acta (1976) 443:629; Szoka & Papahadjopoulos, P.N.A.S. (1978) 75:4194; Kirby, et al., Biotechnology (November 1984) 979-984; Shew, et al., Biochim. Et Biophys. Acta (1985) 816:1). In these passive methods of encapsulation, the passively loaded agent may be membrane associated if hydrophobic, or encapsulated within an entrapped aqueous space if water-soluble. However, the efficiency of loading using passive entrapment during liposome preparation is often quite low.

An advancement in liposome encapsulation techniques was the discovery that drugs which are permeable to the liposomal membrane could be actively loaded into liposomes to concentrations above their equilibrium concentration (i.e., above the concentration that would arise if the drug could freely equilibrate across the liposomal membrane resulting in the same concentration of drug both inside and outside of the liposome). One of the first active loading methods made use of a pH gradient across the liposomal membrane in order to actively load and retain an ionizable agent. In this case, the ionizable drug to be encapsulated exists at least partially in the uncharged form in the external buffer (of one pH) and charged within the altered pH environment of the aqueous interior. The ‘external’ neutral drug can readily cross the liposomal bilayer and become trapped within the aqueous interior due to the pH-dependent conversion of the drug to its charged, membrane-impermeable form.

More recently, an alternative technique was reported for actively encapsulating drugs utilizing an ionophore-based mechanism (Cheung, et al., Biochimica et Biophysica Acta (1998) 1414:205-216). Although this method and the former were applied to the entrapment of a single agent, encapsulation of two agents in a liposome has been met with less success. For example, sequential pH gradient loading of mitoxantrone and vincristine into a single liposome according to the pH-loading method described above resulted in rapid release of entrapped vincristine and 30% loss of mitoxantrone in vivo (Saxon, et al., J Liposome Res. (1999) 9:507-522). In addition, Bally, et al., (U.S. Pat. No. 5,736,155) have described a method of encapsulating two agents by passively loading a first agent followed by actively loading a second agent using a Na+/K+ gradient in conjunction with the ionophore valinomycin. Analysis of liposomes prepared in this manner revealed that although loading of both cytosine arabinoside and methotrexate is possible by this method, only 33% of the available drug is encapsulated. The difficulties lie in the fact that conditions to load and/or retain one agent are independent from that of the other and furthermore that loading conditions required for one agent often lead to poor uptake and/or unfavorable release of a second agent. A need therefore exists to identify mechanisms which allow for the stable uptake, retention and release of more than one therapeutic agent in a single formulation.

In recent work published by the present inventors, a novel active drug-loading technique is described in which liposome-encapsulated metal ions (which establish a transmembrane metal ion gradient) drive the uptake and retention of a variety of drugs into preformed liposomes in which a first drug was passively encapsulated (WO 03/028697). The technique described in WO 03/028697 is one of the first to demonstrate that two or more therapeutic agents can be stably incorporated and retained in a single delivery vehicle and furthermore that active and passive liposome-loading mechanisms can be successfully combined in sequence (provided that the passively-encapsulated drug is entrapped during liposome preparation) to achieve a controllable combination therapy.

The present invention details a method for loading agents (which are typically passively entrapped during preparation of a delivery vehicle), in which these agents are passively loaded into preformed delivery vehicles by selecting conditions whereby said agents can stably permeate and equilibrate across the vehicle membrane. Furthermore, the invention shows that surprisingly, by manipulating loading parameters, additional therapeutic agents may be actively loaded concurrent with the passive loading of the aforementioned agents into these preformed delivery vehicles and achieve substantially higher concentrations inside as compared to outside of the vehicle. It will be appreciated by those knowledgeable in the field that what is surprising is that the conditions required to passively load a therapeutic agent into an already made delivery vehicle (for example, conditions that allow a liposomal membrane to be ‘leaky’ enough for a water-soluble drug to permeate and equilibrate across the membrane) do not result in a collapse of the gradient (generated from the presence of other water-soluble agents) that is required to drive the simultaneous active loading of a second therapeutic agent.

Because bioactive agents utilized in combination therapies typically have very different physico-chemical properties, which results in the need for very different encapsulation conditions, it is not readily apparent in the field that multiple agents in a combination therapy regime could be encapsulated simultaneously into a preformed delivery vehicle. However, the present invention clearly demonstrates that two or more therapeutic agents, with very different physico-chemical properties, can be simultaneously loaded into, as well as retained in, a single delivery vehicle by optimizing loading parameters for the combination of agents rather than for each agent individually. The importance of this is exemplified in combination therapies in which synergistic combinations are preferred. Here, once a synergistic ratio of agents is identified through in vitro screening (as described in WO 03/028696), the agents may be loaded at this ratio into a preformed delivery vehicle which maintains the desired ratio long after administration. The delivery vehicles themselves coordinate release of the encapsulated agents and thus ensure adequate delivery of a non-antagonistic combination of these agents to the target site.

The present co-encapsulation method has numerous advantages over sequential loading that affect the research, development and manufacturing of the resultant pharmaceutical compositions. For example, the extrusion of liposomes during their preparation can be performed in the absence of therapeutic agents which are often toxic. Also, the drug:drug ratios in the final composition can be more precisely controlled leading to superior therapeutic efficacy. Furthermore, the one-step loading process results in reduced time, labour and expense needed for generating a combination therapy, as well as reduced safety risk, increased quality control, increased reproducibility and ease of manufacture.

### DISCLOSURE OF THE INVENTION

The present invention is based on the discovery that simultaneous encapsulation of multiple therapeutic agents into a preformed delivery vehicle can be achieved by utilizing both passive and active loading techniques. More particularly, the method involves at least one agent equilibrating across the delivery vehicle membrane while another agent concentrates inside the delivery vehicle.

Thus, in one aspect, the invention relates to a method for simultaneously loading at least two therapeutic agents into a preformed delivery vehicle wherein two mechanistically different loading techniques are utilized.

The invention further relates to a method for simultaneously loading at least two therapeutic agents into a preformed delivery vehicle wherein at least one agent is passively loaded and at least a second agent is actively loaded. In this case, loading is carried out under conditions such that the passively loaded agent can readily permeate and equilibrate across the membrane without dissipating the gradient required to drive the active encapsulation of a second agent.

Preferably, lipid-based delivery vehicles are utilized.

Even more preferably, liposomes are used for the purpose of the invention.

More particularly, the invention relates to a method for simultaneously loading at least two therapeutic agents, preferably anticancer agents, into a delivery vehicle, wherein the agents are present in the vehicles at ratios that are synergistic or additive (i.e., non-antagonistic). Prior to encapsulation, the ratios of therapeutic agents in the combination are selected so that the combination exhibits synergy or additivity on cells tested in vitro.

In another aspect, the invention relates to a composition which comprises delivery vehicles, said delivery vehicles having encapsulated therein at least two therapeutic agents wherein the agents are encapsulated by carrying out the methods of the invention.

In another aspect, the invention relates to a composition for parenteral administration comprising two or more agents encapsulated in the vehicle composition wherein the agents are encapsulated using the methods of the invention at a ratio that is synergistic or additive. Since the pharmacokinetics of the composition are controlled by the delivery vehicles themselves, encapsulation in a single delivery vehicle allows two or more agents to be delivered to the disease site in a coordinated fashion, thereby assuring that the agents will be present at the disease site at the desired non-antagonistic ratio.

### BRIEF DESCRIPTION OF THE DRAWINGS

FIG. 1A is a graph showing simultaneous loading of cytarabine (▪) and daunorubicin (□) at 40° C. into DSPC/DSPG/Cholesterol (70:20:10 mole ratio) liposomes as a function of time using 100 mM Cu(II)gluconate buffered to pH 7.4 with triethanolamine (TEA) as the internal medium and 300 mM sucrose, 20 mM sodium phosphate, 1 mM ethylenediaminetetraacetic acid (EDTA), pH 7.4 as the external medium. Loading was carried out at a drug-to-lipid mole ratio of 0.04:1 for daunorubicin and a drug concentration of 200 μmol/mL for cytarabine.

FIG. 1B is a graph showing simultaneous loading of cytarabine (▪) and daunorubicin (□) at 50° C. into DSPC/DSPG/Cholesterol (70:20:10 mole ratio) liposomes as a function of time using 100 mM Cu(II)gluconate buffered to pH 7.4 with TEA as the internal medium and 300 mM sucrose, 20 mM sodium phosphate, 1 mM EDTA, pH 7.4 as the external medium. Loading was carried out at a drug-to-lipid mole ratio of 0.04:1 for daunorubicin and a drug concentration of 200 μmol/mL for cytarabine.

FIG. 1C is a graph simultaneous loading of cytarabine (▪) and daunorubicin (□) at 60° C. into DSPC/DSPG/Cholesterol (70:20:10 mole ratio) liposomes as a function of time using 100 mM Cu(II)gluconate buffered to pH 7.4 with TEA as the internal medium and 300 mM sucrose, 20 mM sodium phosphate, 1 mM EDTA, pH 7.4 as the external medium. Loading was carried out at a drug-to-lipid mole ratio of 0.04:1 for daunorubicin and a drug concentration of 200 μmol/mL for cytarabine.

FIG. 2A is a graph showing loading of cytarabine into DSPC/DSPG/Cholesterol (70:20:10 mole ratio) liposomes as a function of time (during simultaneous loading of daunorubicin) using 25 mM (), 50 mM (▾), 75 mM (▪) and 100 mM (♦) Cu(II)gluconate buffered to pH 7.4 with TEA as the internal medium and 300 mM sucrose, 20 mM sodium phosphate, pH 7.4 as the external medium. Loading was carried out at 50° C. at a drug-to-lipid mole ratio of 0.1:1 for daunorubicin and a drug concentration of 200 μmol/mL for cytarabine.

FIG. 2B is a graph showing the percent encapsulation of daunorubicin in DSPC/DSPG/Cholesterol (70:20:10 mole ratio) liposomes as a function of time (during simultaneous loading of cytarabine) using 25 mM (), 50 mM (▾), 75 mM (▪) and 100 mM (♦) Cu(II)gluconate buffered to pH 7.4 with TEA as the internal medium and 300 mM sucrose, 20 mM sodium phosphate, pH 7.4 as the external medium. Loading was carried out at 50° C. at a drug-to-lipid mole ratio of 0.1:1 for daunorubicin and a drug concentration of 200 μmol/mL for cytarabine.

FIG. 3 is a graph showing simultaneous loading of FUDR (∘) and daunorubicin () into DSPC/DSPG/Cholesterol (70:20:10 mole ratio) liposomes as a function of time using 100 mM Cu(II)gluconate buffered to pH 7.0 with TEA as the internal medium and 300 mM sucrose, 40 mM phosphate, 1 mM EDTA, pH 7.0 as the external medium. Loading was carried out at 50° C. at a drug-to-lipid mole ratio of 0.1:1 for daunorubicin and a drug concentration of 60 μmol/mL for FUDR.

FIG. 4A is a graph showing simultaneous loading of gemcitabine (∘) and doxorubicin () at 37° C. into DSPC/DSPG/Cholesterol (70:20:10 mole ratio) liposomes as a function of time using 100 mM Cu(II)gluconate buffered to pH 7.4 with TEA as the internal medium and HBS, pH 7.4 as the external medium.

FIG. 4B is a graph showing simultaneous loading of gemcitabine (∘) and doxorubicin () at 50° C. into DSPC/DSPG/Cholesterol (70:20:10 mole ratio) liposomes as a function of time using 100 mM Cu(II)gluconate buffered to pH 7.4 with TEA as the internal medium and HBS, pH 7.4 as the external medium.

FIG. 4C is a graph showing simultaneous loading of gemcitabine (∘) and doxorubicin () at 60° C. into DSPC/DSPG/Cholesterol (70:20:10 mole ratio) liposomes as a function of time using 100 mM Cu(II)gluconate buffered to pH 7.4 with TEA as the internal medium and HBS, pH 7.4 as the external medium.

FIG. 5 is a graph showing the daunorubicin-to-cytarabine mol ratio as a function of time during simultaneous loading of the two drugs at room temperature (), 40° C. (∘) and 45° C. (▾) into DMPC/Cholesterol (70:30 mole ratio) liposomes comprising 300 mM citrate, pH 4.0 as the internal medium and HBS, pH 7.4 as the external medium.

FIG. 6 is a graph showing the FUDR-to-cpt-11 mol ratio as a function of time during simultaneous loading of the two drugs at 50° C. into DSPC/Cholesterol (70:30 mole ratio) liposomes comprising 100 mM Cu(II)gluconate buffered to pH 7.0 with TEA as the internal medium and SPE, pH 7.0 as the external medium.

### MODES OF CARRYING OUT THE INVENTION

The method of the invention involves simultaneous loading of at least two therapeutic agents into preformed delivery vehicles wherein two mechanistically different loading techniques are utilized. The method further involves the simultaneous loading of at least two agents into preformed delivery vehicles wherein at least one agent is passively loaded and at least a second agent is actively loaded.

Preferably, a passive and an active loading technique are used simultaneously to load at least two different therapeutic agents.

More preferably, the therapeutic agents are loaded at a ratio determined to be non-antagonistic.

Even more preferably, the therapeutic agents are retained in the delivery vehicle for an amount of time sufficient to ensure delivery of adequate concentrations of the agents to the site of desired activity.

Delivery vehicles may include lipid carriers, liposomes, cyclodextrins, and the like. Liposomes can be prepared as described in Liposomes: Rational Design (A. S. Janoff ed., Marcel Dekker, Inc., N.Y.), or by additional techniques known to those knowledgeable in the art. Liposomes of the invention may contain therapeutic lipids, which include ether lipids, phosphatidic acid, phosphonates, ceramide and ceramide analogues, sphingosine and sphingosine analogues and serine-containing lipids. Liposomes may also be prepared with surface stabilizing hydrophilic polymer-lipid conjugates such as polyethylene glycol-DSPE, to enhance circulation longevity. The incorporation of negatively charged lipids such as phosphatidylglycerol (PG) and phosphatidylinositol (PI) may also be added to liposome formulations to increase the circulation longevity of the carrier. These lipids may be employed to replace hydrophilic polymer-lipid conjugates as surface stabilizing agents. Embodiments of this invention may make use of low-cholesterol liposomes containing PG or PI to prevent aggregation thereby increasing the blood residence time of the carrier.

Cyclodextrins comprise cavity-forming, water-soluble, oligosaccharides that can accommodate water-insoluble drugs in their cavities. Cyclodextrins can be prepared using procedures known to those skilled in the art. For example, see Atwood, et al., Eds., “Inclusion Compounds,” Vols. 2 & 3, Academic Press, NY (1984); Bender, et al., “Cyclodextrin Chemistry,” Springer-Verlag, Berlin (1978); Szeitli, et al., “Cyclodextrins and Their Inclusion Complexes,” Akademiai Kiado, Budapest, Hungary (1982) and WO 00/40962.

Preferably, liposomes are used for the practice of the invention.

More preferable, liposomes less than 500 nm are employed.

Even more preferably, liposomes less than 200 m are used.

Liposomes for use in this invention may be prepared to be of “low-cholesterol.” Such liposomes contain an amount of cholesterol that is insufficient to significantly alter the phase transition characteristics of the liposome (typically less than 20 mol % cholesterol). The incorporation of less than 20 mol % cholesterol in liposomes can allow for retention of drugs not optimally retained when liposomes are prepared with greater than 20 mol % cholesterol. Additionally, liposomes prepared with less than 20 mol % cholesterol display narrow phase transition temperatures, a property that may be exploited for the preparation of liposomes that release encapsulated agents once administered due to the application of heat (thermosensitive liposomes).

By “loaded” or “encapsulation”, it is meant stable association with the delivery vehicle. Thus, it is not necessary for the vehicle to surround the agents as long as the agents are stably associated with the vehicles when administered in vivo. Thus, “stably associated with” and “loaded in” or “loaded with” or “co-loaded with” or “dual-loaded” or “encapsulated in” or “encapsulated with” or “co-encapsulated in or with” are intended to be synonymous terms. They are used interchangeably in this specification.

Loading of an agent is established by incubation of the drugs and delivery vehicles at a suitable temperature after addition of the agent to the external medium. Loading may be affected by a variety of parameters including, but not limited to, temperature, pH, ionic strength, pressure, solvents, surfactants and radio-frequency waves.

Techniques for loading are also dependent on the nature of the delivery vehicles and/or the therapeutic agents. For example, loading of one drug combination into a particular liposomal formulation may require a specific temperature range to ensure adequate loading of the drug combination; whereas a different liposomal formulation may require a particular transmembrane gradient in allowing for adequate dual-loading of multiple agents. Alternatively, dual-loading of one drug combination may be differentially affected by, for example, ionic strength as compared to another drug combination.

Typically, passive methods of encapsulating agents involve encapsulating the agent during the preparation of the delivery vehicles. This approach is limited by the solubility of the drugs in aqueous buffer solutions and the large percentage of drug that is not trapped within the delivery system. Techniques to improve loading in liposomes have included co-lyophilizing the drugs with the lipid sample and rehydrating in the minimal volume allowed to solubilize the drugs as well as varying the pH of the buffer, increasing temperature or altering the salt concentration of the buffer. Passive encapsulation in the present invention involves loading the drug after formation of the delivery vehicle. In this case, the drug in solution is mixed with a solution of delivery vehicles and allowed to equilibrate across the separating membrane. Here too, loading can be improved by adjusting temperature, pH, osmolarity, etc. However, surprisingly, by optimizing the loading conditions in the presence of a second therapeutic agent, encapsulation of both agents can be performed simultaneously and loading parameters can be manipulated such that adequate dual-loading can be achieved in a single step. By identifying loading conditions for two agents simultaneously, the ratio of these agents in the final composition can be better controlled. This is important when it is desirable to have a delivery vehicle containing a ratio of agents that is non-antagonistic. It is conceivable that more than two agents could be loaded in this manner and is within the scope of this invention.

Active methods of encapsulating to be used concurrent with this novel passive encapsulation mechanism include, but are not limited to, pH gradient loading or metal-complexation loading. With pH gradient loading, liposomes are formed which encapsulate an aqueous phase of a selected pH. Hydrated liposomes are placed in an aqueous environment of a different pH selected to remove or minimize a charge on the drug or other agent to be encapsulated. Once the drug moves inside the liposome, the pH of the interior results in a charged drug state, which prevents the drug from permeating the lipid bilayer, thereby entrapping the drug in the liposome.

To create a pH gradient, the original external medium can be replaced by a new external medium having a different concentration of protons. The replacement of the external medium can be accomplished by various techniques, such as, by passing the lipid vesicle preparation through a gel filtration column, e.g., a Sephadex® G-50 column, which has been equilibrated with the new medium (as set forth in the examples below), or by centrifugation, dialysis, or related techniques. The internal medium may be either acidic or basic with respect to the external medium.

After establishment of a pH gradient, a pH gradient loadable agent is added to the mixture and encapsulation of the agent in the liposome occurs as described above.

Loading using a pH gradient may be carried out according to methods described in U.S. Pat. Nos. 5,616,341, 5,736,155 and 5,785,987 incorporated herein by reference. A preferred method of pH gradient loading is the citrate-based loading method utilizing citrate as the internal buffer at a pH of 2-6 and a neutral external buffer.

Various methods may be employed to establish and maintain a pH gradient across a liposome all of which are incorporated herein by reference. This may involve the use of ionophores that can insert into the liposome membrane and transportions across membranes in exchange for protons (see for example U.S. Pat. No. 5,837,282). Compounds encapsulated in the interior of the liposome that are able to shuttle protons across the liposomal membrane and thus set up a pH gradient (see for example U.S. Pat. No. 5,837,282) may also be utilized. These compounds comprise an ionizable moiety that is neutral when deprotonated and charged when protonated. The neutral deprotonated form (which is in equilibrium with the protonated form) is able to cross the liposome membrane to exit the liposome and thus leave a proton behind in the interior of the liposome and thereby cause an decrease in the pH of the interior. Examples of such compounds include methylammonium chloride, methylammonium sulfate, ethylenediammonium sulfate (see U.S. Pat. No. 5,785,987) and ammonium sulfate. Internal loading buffers that are able to establish a basic internal pH, can also be utilized. In this case, the neutral form is protonated such that protons are shuttled out of the liposome interior to establish a basic interior. An example of such a compound is calcium acetate (see U.S. Pat. No. 5,939,096).

Metal-based active loading typically uses liposomes with encapsulated metal ions (with or without passively loaded therapeutic agents). Various salts of metal ions are used, presuming that the salt is pharmaceutically acceptable and soluble in an aqueous solution. Actively loaded agents are selected based on being capable of forming a complex with a metal ion and thus being retained when so complexed within the liposome, yet capable of loading into a liposome when not complexed to metal ions. Agents that are capable of coordinating with a metal typically comprise coordination sites such as amines, carbonyl groups, ethers, ketones, acyl groups, acetylenes, olefins, thiols, hydroxyl or halide groups or other suitable groups capable of donating electrons to the metal ion thereby forming a complex with the metal ion. Examples of active agents which bind metals include, but are not limited to, quinolones such as fluoroquinolones and nalidixic acid; anthracyclines such as doxorubicin, daunorubicin and idarubicin; amino glycosides such as kanamycin; and other antibiotics such as bleomycin, mitomycin C and tetracycline; and nitrogen mustards such as cyclophosphamide, thiosemicarbazones, indomethacin and nitroprusside; camptothecins such as topotecan, irinotecan, lurtotecan, 9-aminocamptothecin, 9-nitrocamptothecin and 10-hydroxycamptothecin; and podophyllotoxins such as etoposide. Methods of determining whether coordination occurs between an agent and a metal within a liposome include spectrophotometric analysis and other conventional techniques well known to those of skill in the art.

Liposome loading efficiency and retention properties using metal-based procedures are also dependent on the metal employed and the lipid composition of the liposome. By selecting lipid composition and a metal, loading or retention properties can be tailored to achieve a desired loading or release of a selected agent from a liposome.

Preferably passive loading is used simultaneously with active metal loading in the practice of this invention.

Various combinations of therapeutic agents, having been found to satisfy the criteria of simultaneous loading and retention set forth above, are then provided in the form of formulations of drug delivery vehicles. A “therapeutic agent” is a compound that alone, or in combination with other compounds, has a desirable effect on a subject affected by an unwanted condition or disease.

Certain therapeutic agents are favored for use in combination when the target disease or condition is cancer. Examples are:

“Signal transduction inhibitors” which interfere with or prevents signals that cause cancer cells to grow or divide;

“Cytotoxic agents”;

“Cell cycle inhibitors” or “cell cycle control inhibitors” which interfere with the progress of a cell through its normal cell cycle, the life span of a cell, from the mitosis that gives it origin to the events following mitosis that divides it into daughter cells;

“Checkpoint inhibitors” which interfere with the normal function of cell cycle checkpoints, e.g., the S/G2 checkpoint, G2/M checkpoint and G1/S checkpoint;

“Topoisomerase inhibitors”, such as camptothecins, which interfere with topoisomerase I or II activity, enzymes necessary for DNA replication and transcription;

“Receptor tyrosine kinase inhibitors” which interfere with the activity of growth factor receptors that possess tyrosine kinase activity;

“Apoptosis inducing agents” which promote programmed cell death;

“Antimetabolites,” which closely resemble an essential metabolite and therefore interfere with physiological reactions involving it;

“Telomerase inhibitors” which interfere with the activity of a telomerase, an enzyme that extends telomere length and extends the lifetime of the cell and its replicative capacity;

“Cyclin-dependent kinase inhibitors” which interfere with cyclin-dependent kinases that control the major steps between different phases of the cell cycle through phosphorylation of cell proteins such as histones, cytoskeletal proteins, transcription factors, tumor suppresser genes and the like;

“DNA damaging agents”;

“DNA repair inhibitors”;

“Drug-resistance modulators”;

“Anti-angiogenic agents” which interfere with the generation of new blood vessels or growth of existing blood vessels that occurs during tumor growth; and

“Mitochondrial poisons” which directly or indirectly disrupt mitochondrial respiratory chain function.

Especially preferred combinations for treatment of tumors are the clinically approved combinations outlined in WO 03/028696 and are within the scope of the present invention. As these combinations have already been approved for use in humans, reformulation to assure appropriate delivery is especially important.

### EXAMPLES

The following examples are given for the purpose of illustration and are not by way of limitation on the scope of the invention. Unless otherwise specified, results shown in the drawings are from a single representative example.

Methods for Preparation of Large Unilamellar Liposomes

Lipids were dissolved in chloroform:methanol:water (95:4:1 vol/vol/vol) and subsequently dried under a stream of nitrogen gas and placed in a vacuum pump to remove solvent. Unless otherwise specified, trace levels of radioactive lipid ¹⁴C-DPPC was added to quantify lipid during the formulation process. The resulting lipid film was placed under high vacuum for a minimum of 2 hours. The lipid film was hydrated in the solution indicated to form multilamellar vesicles (MLVs). The resulting preparation was extruded 10 times through stacked polycarbonate filters with an extrusion apparatus (Lipex Biomembranes, Vancouver, BC) to achieve a mean liposome size between 80 and 150 nm. All constituent lipids of liposomes are reported in mole %.

Methods for Quantification of Drug Loading

At various time points after initiation of drug loading, aliquots were removed and passed through a Sephadex® G-50 spin column to separate free from encapsulated drug. To a specified volume of eluant, Triton X-100 or N-ocyl beta-D-glucopyranoside (OGP) was added to solubilize the liposomes. Following addition of detergent, the mixture was heated to the cloud point of the detergent and allowed to cool to room temperature before measurement of the absorbance or fluorescence. Drug concentrations were calculated by comparison to a standard curve. Lipid levels were measured by liquid scintillation counting.

### Example 1

### The Effect of Temperature on Simultaneous Loading of Two Therapeutic Agents

Preformed liposomes were simultaneously loaded with cytarabine (a highly water-soluble drug) and daunorubicin (amphipathic) at three different temperatures. These studies were performed to determine if the loading temperature adversely affected the passive uptake of cytarabine or the active uptake of daunorubicin, or both, into preformed liposomes. Copper ions were employed in the intraliposomal solution to drive the active uptake of daunorubicin.

Cytarabine and daunorubicin were loaded into DSPC/DSPG/Cholesterol liposomes at 40° C., 50° C. and 60° C. Lipid films of DSPC/DSPG/Cholesterol at a mole ratio of 70:20:10 were prepared as described above in the method section. The lipid films were hydrated in 100 mM Cu(II)gluconate adjusted to pH 7.4 with triethanolamine (TEA) and extruded at 70° C. The liposomes were buffer exchanged into 300 mM sucrose, 20 mM phosphate, 1 mM EDTA (SPE), pH 7.4 by crossflow dialysis to remove any copper(II)gluconate from the extraliposomal solution. Prior to addition of drugs, the liposomes were preheated to the appropriate loading temperature for one minute. Cytarabine (with trace amounts of ³H-cytarabine) and daunorubicin were combined then added simultaneously to the pre-heated liposomes. Cytarabine was added at a drug concentration of 200 μmol/mL and daunorubicin was added at a drug-to-lipid mole ratio of 0.04:1. The liposomes and drugs were incubated for one hour to allow for the passive uptake of cytarabine concurrent with the active uptake of daunorubicin. Aliquots were removed and analyzed for drug and lipid content at designated time intervals. The extent of daunorubicin loading was determined spectrophotometrically and cytarabine and lipid levels were determined by liquid scintillation counting.

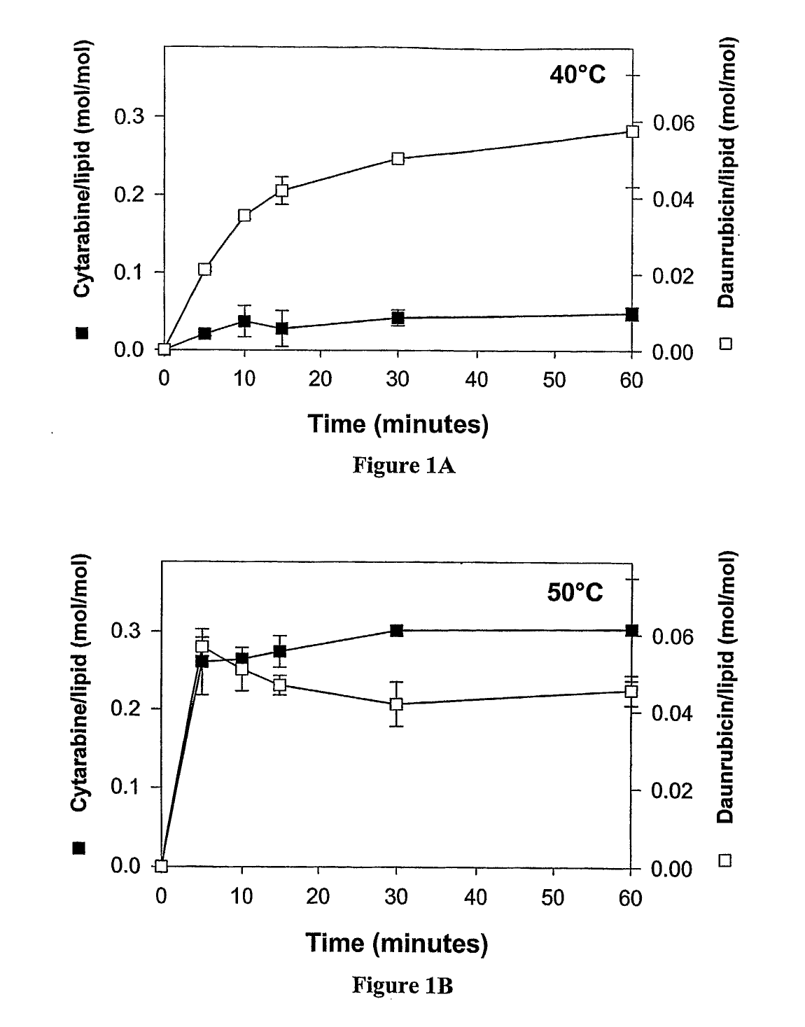

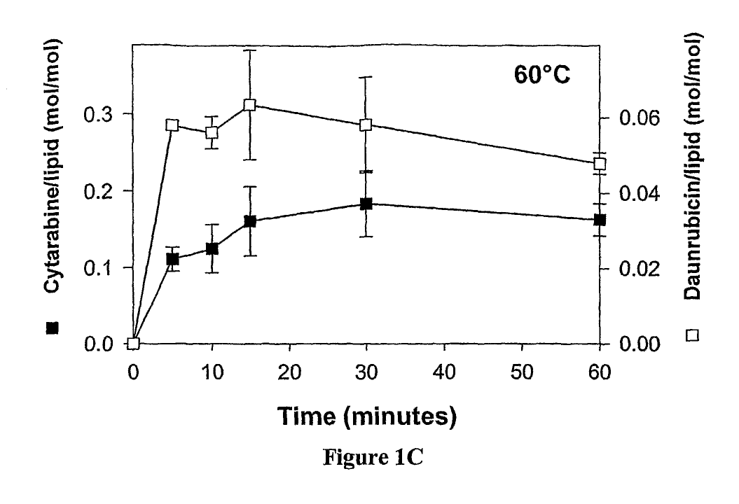

Results summarized in FIG. 1A show that daunorubicin is efficiently loaded and retained in DSPC/DSPG/Cholesterol liposomes at 40° C. in the presence of cytarabine loading. These results also show that passive loading of cytarabine is less than desirable at 40° C. during the simultaneous active metal-loading of daunorubicin.

Conversely, results summarized in FIG. 1B show that at 50° C. both daunorubicin and cytarabine simultaneously load to adequate levels into DSPC/DSPG/Cholesterol liposomes and, importantly, each drug is effectively retained in the liposomes during the course of the experiment.

FIG. 1C shows that cytarabine loading at 60° C. is compromised compared to loading at 50° C. and that although daunorubicin loads at this temperature, it is not well-retained.

Together, these results indicate that loading and retention of both drugs is optimal at 50° C. and, importantly, that temperature affects the uptake and retention of each drug. Also shown in these experiments is that the conditions required for adequate passive uptake of cytarabine (i.e., heating to 50° C. to make the liposomal membrane slightly ‘leaky’ and therefore permeable to cytarabine) did not result in a collapse of the metal ion gradient required to actively load daunorubicin (i.e., loss of copper ions from the intraliposomal solution was negligible—data not shown.)

### Example 2

### The effect of Metal Ion-Gradient Capacity and Osmolality on the Co-Loading of Cytarabine and Daunorubicin

Cytarabine and daunorubicin were simultaneously loaded into liposomes that had been prepared using various internal concentrations of buffered copper gluconate in order to determine the effect of copper gluconate content and osmolarity on drug loading.

Cytarabine and daunorubicin were loaded at 50° C. into pre-formed DSPC/DSPG/Cholesterol liposomes containing either 25 mM, 50 mM, 75 mM or 100 mM Cu(II)gluconate. Lipid films of DSPC/DSPG/Cholesterol at a mole ratio of 70:20:10 were prepared as described above in the method section. The lipid films were hydrated in the designated Cu(II)gluconate concentration and adjusted to pH 7.0 with triethanolamine (TEA). The liposomes were buffer exchanged into 300 mM sucrose, 20 mM sodium phosphate, pH 7.0 by crossflow dialysis to remove any copper(II)gluconate from the extraliposomal solution. Prior to addition of drugs, the liposomes were diluted in SPE and preheated at 50° C. for one minute. Cytarabine (with trace amounts of ³H-cytarabine) and daunorubicin were combined then added simultaneously to the pre-heated liposomes. Cytarabine was added at a drug concentration of 200 μmol/mL and daunorubicin was added at a drug-to-lipid mole ratio of 0.1:1. The liposomes and drugs were incubated for one hour to allow for the passive uptake of cytarabine concurrent with the active uptake of daunorubicin. Aliquots were removed and analyzed for drug and lipid content at designated time intervals. The extent of daunorubicin loading was determined spectrophotometrically and cytarabine and lipid levels were determined by liquid scintillation counting.

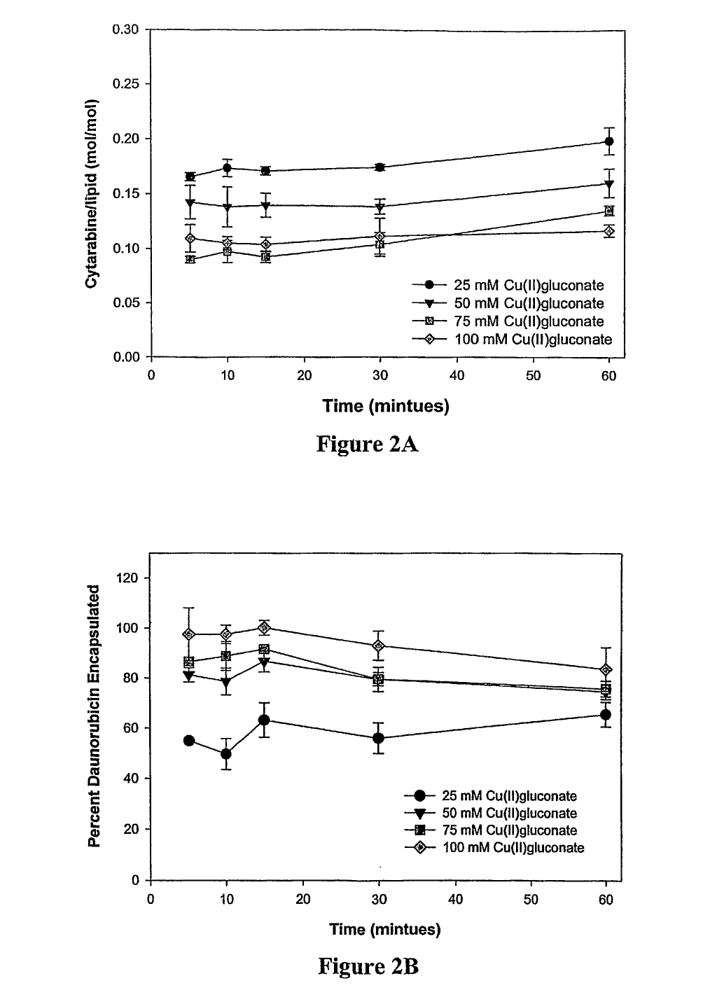

Results summarized in FIG. 2A show that cytarabine loading (during the simultaneous uptake of daunorubicin) is sufficient at the four Cu(II)gluconate concentrations utilized. However, the extent of loading is improved at the two lower copper concentrations as opposed to at 75 mM and 100 mM. Therefore, based on this experiment, there is reason to believe that passive loading of cytarabine can be enhanced by lowering the internal osmolarity.

The graph in FIG. 2B illustrates the corresponding loading of daunorubicin (as percent encapsulation) during the simultaneous uptake of cytarabine seen in FIG. 2A. The data shows that daunorubicin loading is altered under varying internal copper gluconate concentrations. Loading is sufficient at 100 mM, 75 mM and even 50 mM but compromised at 25° C. mM copper gluconate. This data therefore suggests that reducing the copper gluconate gradient may inhibit the uptake of daunorubicin and therefore conditions must be carefully optimized to identify those that allow for adequate loading of both drugs simultaneously.

### Example 3

### Simultaneous Loading of FUDR and Daunorubicin into Preformed Liposomes

Floxuridine (FUDR) and daunorubicin were loaded into preformed liposomes in order to determine whether additional drug combinations of passive and actively loaded drugs could occur simultaneously. As a starting point, dual-loading was carried out under conditions optimized above for cytarabine and daunorubicin. The pH and osmolarity of the intraliposomal solution as well as the loading temperature were manipulated in order to determine conditions which would allow for the stable uptake and retention of both FUDR and daunorubicin (data not shown). The results summarized below demonstrate that loading at 50° C. was optimal for this drug combination, similar to cytarabine/daunorubicin; however, a lower pH and higher copper gluconate concentration were required to load both FUDR and daunorubicin to adequate levels indicating the importance of optimizing conditions for the combination of drugs rather than for each drug individually.

FUDR and daunorubicin were loaded at 50° C. into pre-formed DSPC/DSPG/Cholesterol liposomes (70:20:10) containing 100 mM Cu(II)gluconate adjusted to pH 7.0 with triethanolamine (TEA). The liposomes were buffer exchanged into 300 mM sucrose, 40 mM phosphate, 1 mM EDTA, pH 7.0 by crossflow dialysis to remove any copper(II)gluconate from the extraliposomal solution. Prior to addition of drugs, the liposomes were diluted in SPE and preheated at 50° C. for one minute. FUDR (with trace amounts of ³H-FUDR) and daunorubicin were combined then added simultaneously to the pre-heated liposomes. FUDR was added at a drug concentration of 60 μmol/mL and daunorubicin was added at a drug-to-lipid mole ratio of 0.1:1. The liposomes and drugs were incubated for one hour to allow for the passive uptake of cytarabine concurrent with the active uptake of daunorubicin. Aliquots were removed and analyzed for drug and lipid content at designated time intervals. The extent of daunorubicin loading was determined spectrophotometrically and FUDR and lipid levels were determined by liquid scintillation counting.

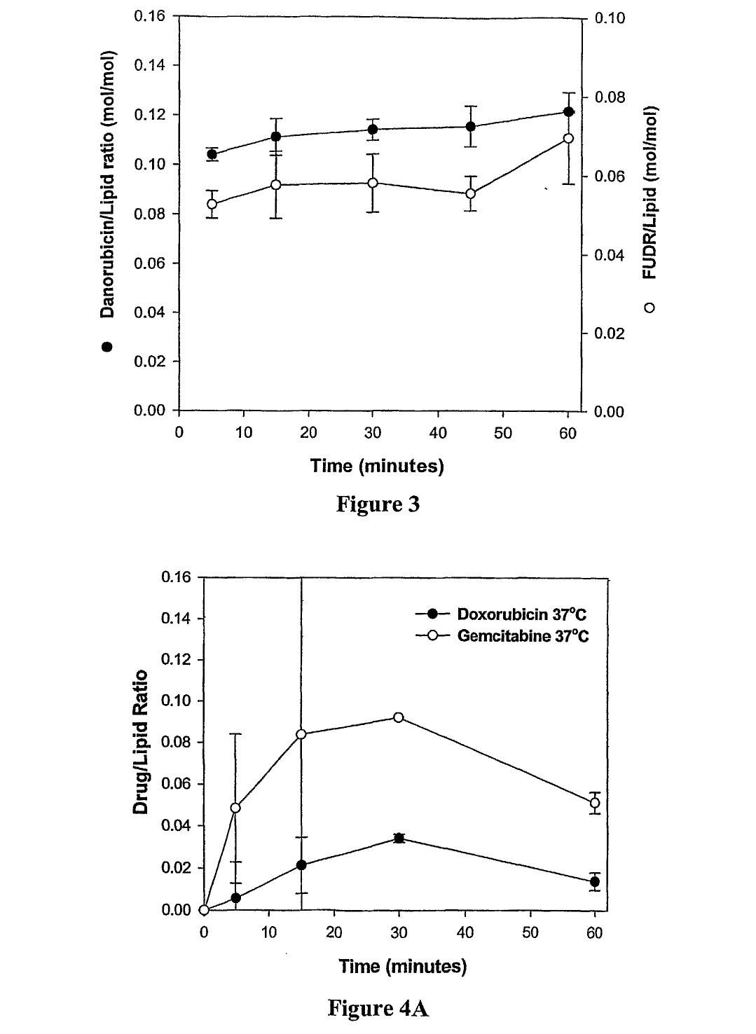

The graph in FIG. 3 shows that both FUDR and daunorubicin were loaded to sufficient levels under unique conditions optimized for this drug combination and, importantly, that each drug was well maintained during the course of the experiment.

### Example 4

### Simultaneous Loading of Gemcitabine and Doxorubicin into Preformed Liposomes

Gemcitabine and doxorubicin were loaded into preformed liposomes in order to further analyze simultaneous loading of additional drug combinations. As a starting point, dual-loading was carried out under conditions optimized above for cytarabine and daunorubicin. The pH and osmolarity of the intraliposomal solution as well as the loading temperature were manipulated in order to determine conditions which would allow for the stable uptake and retention of both gemcitabine and doxorubicin (data not shown). The results summarized below demonstrate that loading at 60° C. was optimal for this drug combination, as opposed to cytarabine/daunorubicin and FUDR/daunorubicin which loaded optimally at 50° C. as detailed above.

Gemcitabine and doxorubicin were loaded into DSPC/DSPG/Cholesterol liposomes at 37° C., 50° C. and 60° C. (Similar loading was also attempted in DMPC/Cholesterol (55:45) liposomes; however, liposomes aggregated therefore no data is reported.) Lipid films of DSPC/DSPG/Cholesterol at a mole ratio of 70:20:10 were prepared as described above. The lipid films were hydrated in 100 mM Cu(II)gluconate, adjusted to pH 7.4 with triethanolamine (TEA) and extruded at 70° C. The liposomes were buffer exchanged into HBS, pH 7.4 by crossflow dialysis. Prior to addition of drugs, the liposomes were preheated to the appropriate loading temperature for one minute. Gemcitabine (with tracer amounts of ³H-gemcitabine) and doxorubicin were combined then added simultaneously to the pre-heated liposomes. Gemcitabine was added to a drug concentration of 30 μmol/mL and doxorubicin was added to a drug concentration of 33 μmol/mL. The liposomes and drugs were incubated for one hour to allow for the passive uptake of gemcitabine concurrent with the active uptake of doxorubicin. Aliquots were removed and analyzed for drug and lipid content at designated time intervals. The extent of doxorubicin loading was determined spectrophotometrically and gemcitabine and lipid levels were determined by liquid scintillation counting.

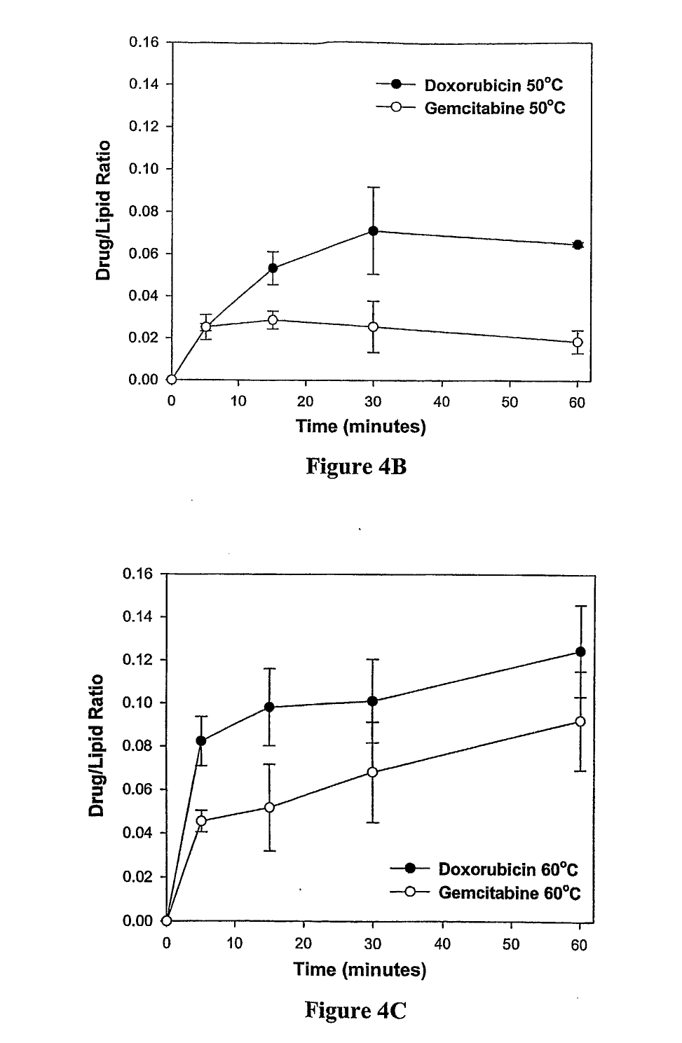

The graph in FIG. 4A shows that although gemcitabine could load effectively at 37° C., the doxorubicin did not load to sufficient levels. In fact, the doxorubicin appeared to aggregate at or below this temperature making it difficult to load and or recover for analysis. In contrast, at 50° C. doxorubicin loading was superior to the gemcitabine loading. The gemcitabine appears to load to minimal levels and then quickly leaks from the liposomes (FIG. 4B). Optimal loading and retention of both drugs occurred at 60° C. as shown in FIG. 4C. There is constant and comparable loading of both drugs over the course of the experiment making it more feasible to load a predetermined ratio of the two drugs.

### Example 5

### Simultaneous Loading of Daunorubicin and Cytarabine in the Presence of Citrate

As described in Example 1, the simultaneous loading of daunorubicin and cytarabine into DSPC/DSPG/Cholesterol (70/20/10) liposomes is temperature dependent. The liposomes of Example 1 contain entrapped copper ions which drive the active uptake of daunorubicin. In order to compare the simultaneous loading and/or temperature dependence of loading of daunorubicin and cytarabine using pH-gradient loading, DMPC/Cholesterol liposomes were prepared in the presence of citrate and the absence of metal ions.

Daunorubicin and cytarabine were loaded at room temperature, 40° C. and 45° C. into DMPC/Cholesterol (70/30 mol ratio) liposomes containing 300 mM citrate, pH 4. Prior to drug loading, the liposomes were buffer exchanged into 20 mM HEPES, 150 mM NaCl (pH 7.4) (HBS) and then diluted to a final lipid concentration of 30 μmol/mL. Cytarabine (with tracer amounts of ³H-cytarabine) and daunorubicin were combined then added simultaneously to the preformed liposomes. Cytarabine was added at a drug concentration of 40 μmol/mL and daunorubicin was added at a drug concentration of 4.5 μmol/mL. The liposomes and drugs were incubated for one hour to allow for the passive uptake of cytarabine concurrent with the active pH-gradient loading of daunorubicin. Aliquots were removed and analyzed for drug and lipid content at designated time intervals. The extent of daunorubicin loading was determined spectrophotometrically and cytarabine and lipid levels were determined by liquid scintillation counting.

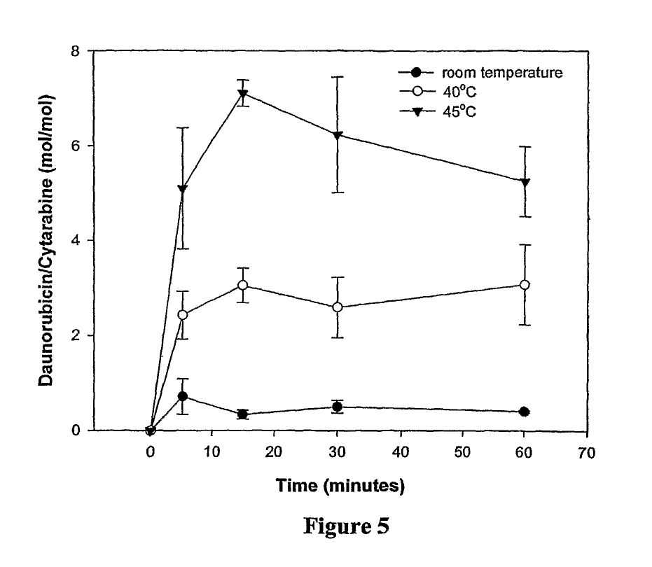

As seen in FIG. 5, the simultaneous loading of daunorubicin and cytarabine using citrate pH-gradient loading is temperature dependent. Loading at 45° C. is more efficient than loading at room temperature or even 40° C. demonstrating that the temperature used for simultaneously loading two drugs must be carefully determined to maximize the uptake of both drugs. These data also shows that other parameters, such as the mechanism used to load one or more drugs (i.e., active metal loading or pH-gradient loading), may be differentially affected by various temperatures.

### Example 6

### Irinotecan and Floxuridine Loading into High-Cholesterol Containing Liposomes

Examples 4 and 5 demonstrate loading of a drug combination into both high (>25%) and low (<25%) cholesterol-containing liposomes. As described in Example 4, simultaneous loading of gemcitabine and doxorubicin was hindered by aggregation of the high cholesterol-containing liposomes (DMPC/Cholesterol; 70:30) though proceeded to sufficient levels in low cholesterol-containing liposomes (DSPC/DSPG/Cholesterol; 70:20:10). In contrast, daunorubicin and cytarabine were able to load simultaneously into DMPC/Cholesterol (70:30) liposomes as described in Example 5. In order to determine if high cholesterol content would affect the simultaneous loading of irinotecan (cpt-11) and floxuridine (FUDR); both drugs were incubated with DSPC/Cholesterol (55:45) liposomes and loading was monitored. The present investigators have previously found that loading is sufficient in low cholesterol-containing DSPC/DSPG/Cholesterol liposomes (data not shown.)

The simultaneous loading of daunorubicin and cytarabine into DSPC/DSPG/Cholesterol (70/20/10) liposomes is temperature dependent. The liposomes of Example 1 contain entrapped copper ions which drive the active uptake of daunorubicin. In order to compare the simultaneous loading and/or temperature dependence of loading of daunorubicin and cytarabine using pH-gradient loading, DMPC/Cholesterol liposomes were prepared in the presence of citrate and the absence of any metal ions.

Lipid films of DSPC/Cholesterol at a mole ratio of 55:45 were prepared as previously described and then hydrated in 100 mM Cu(II)gluconate, adjusted to pH 7.0 with triethanolamine (TEA). The liposomes were buffer exchanged into SPE, pH 7.0. Prior to addition of drugs, the liposomes were preheated to 50° C. for one minute. FUDR (with tracer amounts of ³H-FUDR) and cpt-11 were combined then added simultaneously to the pre-heated liposomes. FUDR was added to a final drug concentration of 110 μmol/mL and cpt-11 was added to a final drug concentration of 7.5 μmol/mL. The liposomes and drugs were incubated for one hour to allow for the passive uptake of FUDR concurrent with the active uptake of Irinotecan. Aliquots were removed and analyzed for drug and lipid content at designated time intervals. The extent of cpt-11 loading was determined spectrophotometrically and FUDR and lipid levels were determined by liquid scintillation counting.

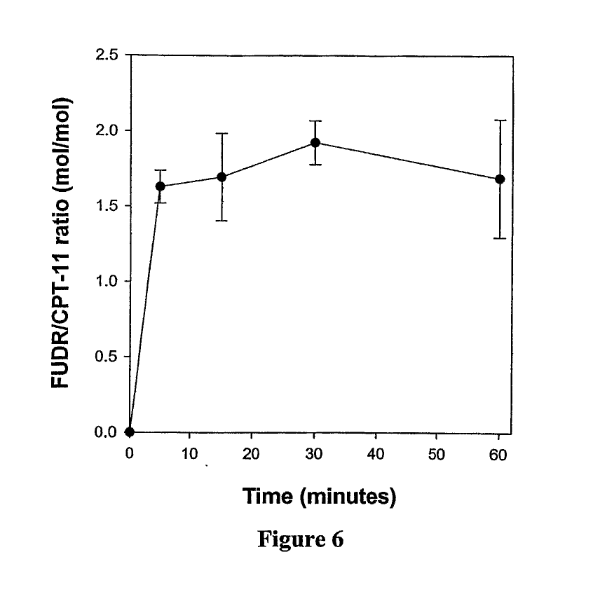

The drugs were effectively loaded at 50° C. and the graph in FIG. 6 shows that the drug ratio of FUDR-to-cpt-11 could be maintained at a sufficient level during the course of the experiment.

1. A method for simultaneously loading at least two agents into the interior of preformed delivery vehicles, which method comprises contacting preformed liposomes with at least a first agent under conditions that effect active loading of said first agent and
simultaneously contacting said preformed liposomes with at least a second agent under conditions that effect passive loading of said second agent.

2. The method of claim 1, which further comprises manipulating the loading temperature to determine an optimum temperature for said loading.

3. The method of claim 1 or 2, wherein said conditions that effect active loading of the first agent include a pH gradient.

4. The method of claim 1 or 2, wherein the conditions that effect active loading of the first agent comprise inclusion of a metal ion in the interior of said preformed delivery vehicles.

5. The method of claim 4, wherein said first agent is able to form a complex with a metal ion.

6. The method of claim 3, wherein the interior of said preformed delivery vehicles is maintained at a pH wherein said first agent is a charged moiety.

7. The method of claim 4 wherein the first agent is a quinolone, an anthracycline, and aminoglycoside, an antibiotic, a nitrogen mustard, a camptothecin or a podophyllotoxin.

8. The method of claim 7, wherein the first agent is daunorubicin, doxorubicin or irinotecan and the second agent is cytarabine, FUDR or gemcitabine.

9. The method of any of claims 1-8 wherein the delivery vehicles are liposomes.

10. The method of any of claims 1-9 wherein the first and second agents are loaded in a non-antagonistic ratio.

11. A composition comprising delivery vehicles loaded by the method of any of claims 1-9.
